(19) United States
(12) Patent Application Publication
(10) Pub. No.: US 2020/0352191 A1
(43) Pub. Date: Nov. 12, 2020

(54) EDIBLE FORMULATIONS AND USES THEREOF

(71) Applicant: Karl Lonsbery, Harrow (CA)

(72) Inventor: Karl Lonsbery, Harrow (CA)

(21) Appl. No.: 16/867,605

(22) Filed: May 6, 2020

Related U.S. Application Data
(60) Provisional application No. 62/843,726, filed on May 6, 2019.

(51) Int. Cl. A23D 7/01; A23D 7/00; A23P 30/40; A23L 33/105; A61K 31/05

(52) U.S. Cl. CPC A23D 7/011; A23D 7/003; A23V 2002/00; A23L 33/105; A61K 31/05; A23P 30/40

(57) ABSTRACT

Edible formulations are provided that include an aqueous component, a lipid component including a triglyceride, and a cannabis-derived oil. The edible formulation can be in the form of an emulsion having a continuous phase including the aqueous component and a dispersed phase including the lipid component and the cannabis-derived oil. Upon whipping or dispensing the edible formulation from a pressurized container, the edible formulation can take the form of a colloid having a continuous phase including the edible formulation and a dispersed phase including a gas. Dairy and nondairy whipped cream products can be provided thereby that exhibit antioxidant properties, provide relaxing and calming effects, and have improved mouthfeel, emulsification properties, and creaminess.

### CROSS-REFERENCE TO RELATED APPLICATIONS

This application claims the benefit of U.S. Provisional Application No. 62/843,726, filed on May 6, 2019. The entire disclosure of the above application is incorporated herein by reference.

### FIELD

The present technology relates to edible formulations for dairy and nondairy whipped cream products that include oil obtained from Cannabis, being a genus of flowering plants in the family Cannabaceae; where more particularly, such formulations are packaged into pressurized containers for dispensing a foamed product therefrom having improved mouthfeel and calming properties.

### INTRODUCTION

This section provides background information related to the present disclosure which is not necessarily prior art.

Various edible formulations can be used to enhance the flavor and appearance of a primary foodstuff. Whipped cream is a widely popular culinary accent that can transform everyday food and beverages, such as desserts and coffees, into exceptional and delightful treats. Often whipped cream is used in a final step in the preparation of another foodstuff or beverage, where whipped cream can be added upon preparation completion or shortly before consumption of the primary foodstuff or beverage. Production of whipped cream, however, can be labor intensive and difficult with respect to timing in preparation of the primary foodstuff or beverage to which it is applied as an accent. For example, whipped cream has a limited shelf life, can spoil without sufficient refrigeration, and can naturally begin to separate into liquid and air components only a short time after it is prepared and whipped into a foamy state. As an alternative to producing whipped cream on demand, disposable pressurized whipped cream dispensing containers can be used. Such containers can include a combination of a cream mixture with an aerosol propellant of pressurized nitrous oxide. As used herein, “whipped cream” includes foamed or whipped dairy-based cream products as well as non-dairy whipped cream products, also referred to in the art as imitation whipped cream or whipped toppings. Such non-dairy whipped cream products can be important for consumers having certain dietary restrictions, including consumers having milk allergies, following vegan diets, and/or for religious reasons.

Important considerations related to the enjoyment of whipped cream include a creamy mouthfeel, richness, flavor impact, and sweetness. These attributes of whipped cream can vary based upon the ingredients employed, the mixing and emulsifying methods used, and the final whipping or foaming methods selected. For example, various ingredients including fats or oils, emulsifiers, sweeteners, and thickeners can be blended, substituted, and exchanged in formulating a dairy versus non-dairy whipped cream. The selection of fats and oils and emulsification thereof into the whipped cream product can further take on additional considerations when certain functional oils and essential oils are included in the formulation. One or more of such functional and/or essential oils can be selected to improve mouthfeel, aroma and taste, and can impact health considerations by including the use of monounsaturated or polyunsaturated fat content, for example. Certain oils can also provide desirable biological effects, including relaxing and calming effects that can serve to reduce stress and anxiety and foster a general feeling of contentment or well-being.

Various oils and blends of oils can be prepared from Cannabis, which is a genus of flowering plants in the family Cannabaceae. The number of species within the genus is unsettled, but three species are typically recognized: Cannabis sativa, Cannabis indica, and Cannabis ruderalis. The genus is indigenous to and originates from Central Asia. The plant is also known as hemp, although this term is often used to refer only to varieties of Cannabis cultivated for non-drug use. Cannabis has long been used for hemp fiber, hemp seeds and their oils, hemp leaves for use as vegetables and as juice, medicinal purposes, and as a recreational drug. Some Cannabis strains have been selectively bred to produce minimal levels of tetrahydrocannabinol (THC), the principal psychoactive constituent. Medical cannabis (or medical marijuana) often refers to the use of cannabis and its constituent cannabinoids to treat disease or alleviate symptoms. For example, cannabis can be used to reduce nausea and vomiting during chemotherapy, to improve appetite in people with HIV/AIDS, and to treat chronic pain and muscle spasms. Cannabinoids derived from cannabis are also under investigation for their potential to affect stroke. Certain applications of cannabis and cannabinoid oil are reported to promote relaxation, alleviate anxiety, and mitigate pain symptoms.

Cannabis-derived oils include cannabinoids, which are a class of diverse chemical compounds that act on cannabinoid receptors in cells that can alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially). One notable cannabinoid, as mentioned above, is the phytocannabinoid THC, the primary psychoactive compound in cannabis. Cannabidiol (CBD) is another major constituent of the cannabis plant. There are at least 113 different cannabinoids isolated from cannabis, which can exhibit varied effects individually and in combination. Cannabinoids, including CBD, have been identified as having antioxidant and neuroprotective properties. These effects appear to take place through the triggering of the cannabinoid receptors in the endocannabinoid system. Cannabinoids may be a potential therapeutic agent for the treatment of oxidative neurological disorders, such as cerebral ischaemia. As described in U.S. Pat. No. 6,630,507 to Hampson et al., which is incorporated herein by reference, cannabinoids have various antioxidant properties that make cannabinoids useful in the treatment and prophylaxis of a wide variety of oxidation associated diseases, such as ischemic, age-related, inflammatory, and autoimmune diseases. Cannabinoids are further described in U.S. Pat. No. 6,630,507 to Hampson et al. as having particular application as neuroprotectants, for example in limiting neurological damage following ischemic insults, such as stroke and trauma, or in the treatment of neurodegenerative diseases, such as Alzheimer's disease, Parkinson's disease, and HIV dementia.

Thus, it would be desirable to have ways of optimizing the production and enjoyment of an edible formulation, such as a whipped cream product, that is formulated in conjunction with one or more cannabis-derived oils, therefore possessing the certain antioxidant properties of cannabinoids, such as CBD, where such combinations can provide relaxing and calming effects while further improving the mouthfeel, emulsification properties, and creaminess of whipped cream product formulations.

### SUMMARY

The present technology includes articles of manufacture, systems, and processes that relate to edible formulations for dairy and nondairy whipped cream products including a cannabis-derived oil, such as cannabidiol, that have improved mouthfeel and emulsification properties and that can further provide relaxing and calming effects to a consumer.

Edible formulations are provided that include an aqueous component, a lipid component including a triglyceride, and a cannabis-derived oil. The edible formulation can be in the form of an emulsion having a continuous phase including the aqueous component and a dispersed phase including the lipid component and the cannabis-derived oil. The edible formulation can be in the form of a colloid having a continuous phase including the edible formulation and a dispersed phase including a gas, such as nitrous oxide. Ways of making such edible formulations include providing an edible formulation including an aqueous component, a lipid component including a triglyceride, and a cannabis-derived oil, and generating a colloid having a continuous phase including the edible formulation and a dispersed phase including a gas. The edible formulation can take the form of a dairy or a nondairy whipped cream product.

Edible formulations are provided that include water, a fat, including a triglyceride, and a cannabinoid oil, where examples of cannabinoid oil include one or more various cannabinoids, such as tetrahydrocannabinol (THC) and/or cannabidiol (CBD). Such formulations can further include a sweetener, a flavoring agent, and/or a colorant. The fat can include a vegetable-based fat and/or an animal-based fat, such as cream. The edible formulation can be packaged in a pressurized container, where the pressurized container can be pressurized with nitrous oxide. The cannabinoid oil can have less than about 0.3% THC and in certain embodiments the the cannabinoid oil can have about 0.0% THC. The cannabinoid oil can include about 70-90% CBD. Various methods of making such edible formulations are provided.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 schematically depicts two-dimensional chemical formulae representing esterification of glycerol and three fatty acids in forming a triglyceride, where R′, R″, and R″′ represent saturated or unsaturated aliphatic chains that can be the same or different.

FIG. 2 schematically depicts a portion of a phospholipid layer about a globule of triglycerides, forming an emulsion having a dispersed phase, where a continuous aqueous phase including water molecules surrounds the globule.

FIG. 3 schematically depicts aeration of an edible formulation where partially crystalline fat globules coalesce about a gas bubble, where proteins associated with the fat globules (e.g., whey, casein) assist in stabilizing the fat globules and in forming a membrane about the gas bubble.

FIG. 4 schematically depicts aeration of an edible formulation where multiple partially crystalline fat globules including triglycerides are coalesced about multiple gas bubbles forming a colloid having a dispersed phase of gas bubbles surrounded by the partially crystalline fat globules, which are further surrounded by a continuous aqueous phase.

FIG. 5 includes a series of representative scanning electron micrographs of an aerated edible formulation in the form of whipped cream, where panel A shows the relative size and prevalence of air bubbles (a) and fat globules (f) (the scale bar representing 30 micrometers), panel B shows the internal structure of an air bubble, showing a layer of partially coalesced fat that has stabilized the air bubble (the scale bar representing 5 micrometers), and panel C provides detail of the partially coalesced fat layer, showing the interaction of the individual fat globules (the scale bar representing 3 micrometers).

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on. As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

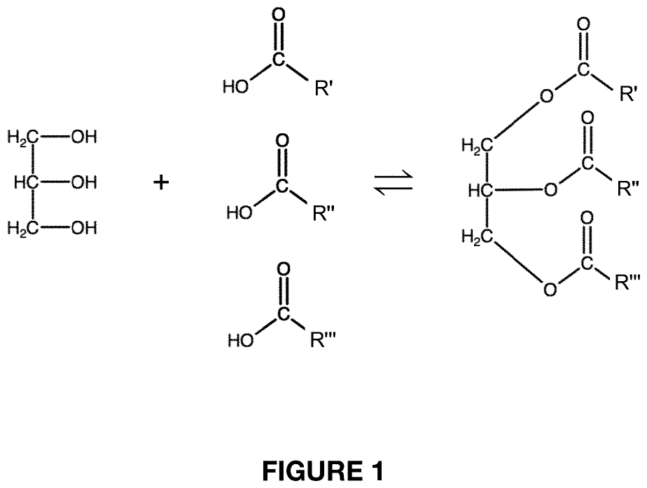

The present technology provides various edible formulations including certain formulations useful for producing dairy and nondairy whipped cream products including a cannabis-derived oil, where the resulting whipped cream products have improved mouthfeel and emulsification properties and provide relaxing and calming effects to a consumer. Edible formulations can include an aqueous component, a lipid component including a triglyceride, and a cannabis-derived oil. The edible formulations can include dairy-based lipids or fats, for dairy-based whipped cream products, or nondairy-based lipids or fats for nondairy whipped cream, also referred to as imitation whipped cream or whipped topping. Either of the dairy-based or non-dairy based lipids can be blended with cannabis-derived oil, where additional ingredients can be included, such as sweeteners, emulsifiers, flavorings, etc. The cannabis-derived oil can work in concert with the dairy and/or nondairy lipids to improve the mouthfeel, creaminess, emulsification, and/or colloidal characteristics of the resulting product. The cannabis-derived oil can be extracted from cannabis and can include various cannabinoids, such as tetrahydrocannabinol (THC) and/or cannabidiol (CBD) oil, which can include various other compounds derived from cannabis, such as terpenes. The edible composition can therefore provide antioxidant properties of cannabinoids as well as calming and stress-reducing effects associated therewith, improving enjoyment and relaxation of the consumer.

Where the edible formulation is to be formulated as a whipped cream, the final whipped cream product is in a foam-like state, being a colloidal suspension of gas bubbles in liquid continuous phase, including the aqueous and lipid components. Unlike egg-based foams, which are stabilized by protein, whipped cream is stabilized by its own fat content. Milk fat is a complex mixture of lipids, but the most prevalent one is triglycerides, made by combining three fatty acids (that's the “tri-” part) and glycerol (that's the “glyceride” part). Fatty acids include carboxylic acids with long carbon chains attached. Carboxylic acids are a class of carbon containing acids in which a carbon is connected to an oxygen atom by a double bond, and an oxygen-hydrogen grouping by a single bond. An example of glycerol esterified with three fatty acids to form a triglyceride is shown in FIG. 1, where R′, R″, and R″′ indicate carbon chains that can be the same or different. Carbon chains R′, R″, and R″′ can be of various lengths, can be fully saturated (carbon-carbon single bonds), monounsaturated (one instance of a carbon-carbon double bond), and polyunsaturated (multiple instances of carbon-carbon double bonds). Glycerol is a polyol, particularly a triol, that can be esterified with one, two, or three fatty acids, including one or more of the same or different fatty acids, as depicted in FIG. 1. As fatty acids are added to glycerol, a monoglyceride, a diglyceride, and a triglyceride are subsequently formed.

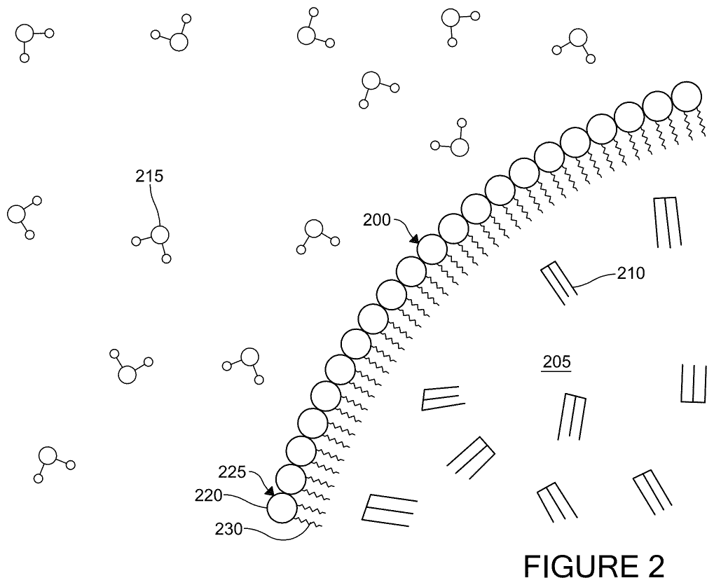

Fat and water do not mix, but triglycerides can be protected by layers or membranes of phospholipids, depending on the source, where phospholipids are special biological molecules that possess hydrophilic (water-loving) and hydrophobic (water-fearing) regions. FIG. 2 schematically depicts a portion of a phospholipid layer 200 about a globule 205 including triglycerides 210, forming an emulsion having a dispersed phase, where an aqueous continuous phase including water molecules 215 surrounds the globule 205. Along with the triglycerides 210, the globule 205 can include the cannabis-derived oil. As depicted in FIG. 2, a hydrophilic head 220 of each phospholipid 225 faces the water molecules 215, forcing the hydrophobic tails 230 to gather around the fatty triglycerides 210 and one or more cannabis-derived oils, which form the globule 205 of lipid dispersed in the aqueous continuous phase. Many globules 205 can form in this manner.

When a liquid containing an emulsion of a lipid component and an aqueous component is aerated (e.g., when dairy cream is whipped using a whisk), a couple of things happen. First, air is forcibly integrated into the cream, forming bubbles of gas that pop almost as quickly as they form; the surface tension of the cream simply is not strong enough to entrap them. But, after a few more minutes of being knocked around, fat globules in the cream begin to destabilize as their protective phospholipid membranes are broken apart by the force of the whisk. This exposes portions of the hydrophobic triglycerides, causing them seek each other out and stick together. However, some of these exposed areas of fat may not find another triglyceride to associate with, and because they would rather associate with anything but hydrophilic water molecules, the triglycerides align themselves with fairly neutral pockets of air. A network of fat globule-surrounded air bubbles develops and the stable, somewhat solid and colloidal structure known as whipped cream results. As the triglycerides can operate as an emulsifier to form the desired emulsion, it is necessary that there are a certain amount of triglycerides in the formulation—whipped cream typically cannot form with anything with lower than a 30% content of triglycerides and other emulsifiers.

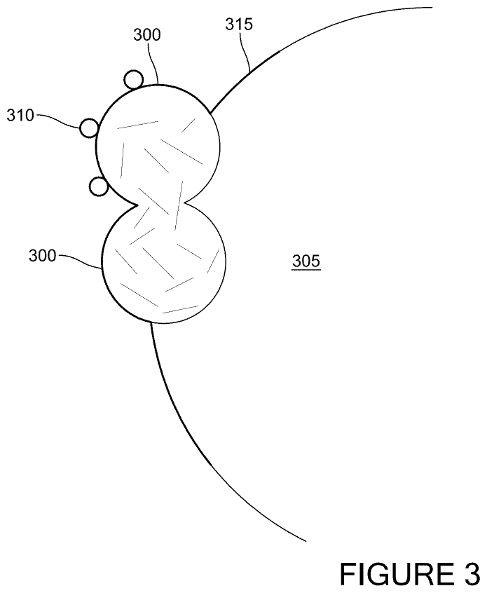

The structure of whipped cream is very similar to the fat and air structure that exists in ice cream. Cream is an emulsion with a fat content of about 35-40 wt. %. When a bowl of heavy cream is whipped, the agitation and the air bubbles that are added into the liquid cause the fat globules to begin to partially coalesce in chains and clusters and adsorb to and spread around the air bubbles. This effect is schematically depicted in FIG. 3, which schematically depicts aeration of an edible formulation where partially crystalline fat globules 300 coalesce about a gas bubble 305, where proteins associated with the fat globules 300 (e.g., whey 310, casein micelles 315) assist in stabilizing the fat globules 300 in forming a membrane about the gas bubble 305.

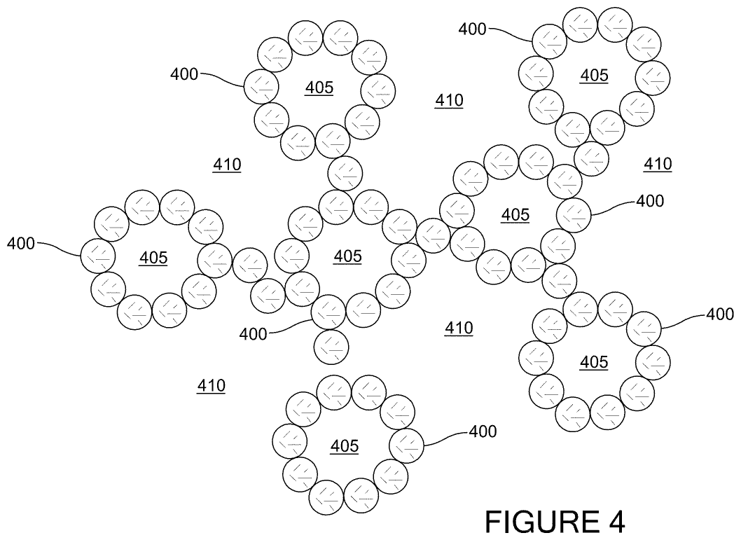

As the fat partially coalesces, it causes one fat-stabilized air bubble to be linked to the next, and so on. The whipped cream soon starts to become stiff and dry appearing and takes on a smooth texture. This results from the formation of this partially coalesced fat structure stabilizing the air bubbles. The aqueous component, including water, lactose, and proteins, is trapped in the space around the fat-stabilized air bubbles. The crystalline fat content is essential (hence whipping of cream can be very temperature dependent) so that the fat globules partially coalesce into a 3-dimensional structure rather than fully coalesce into larger and larger globules that are not capable of structure-building. This is caused by the crystals within the globules that cause the globules to stick together into chains and clusters, but still allow the globules to substantially retain their individual identity and structure. An example is shown in FIG. 4, which schematically depicts aeration of an edible formulation where multiple partially crystalline fat globules 400 including triglycerides are coalesced about multiple gas bubbles 405 forming a colloid having a dispersed phase of gas bubbles 405 surrounded by the partially crystalline fat globules 400, which are further surrounded by a continuous aqueous phase 410. However, if whipped cream is whipped too far, the colloidal structure will collapse as the fat begins to churn and butter particles begin to form.

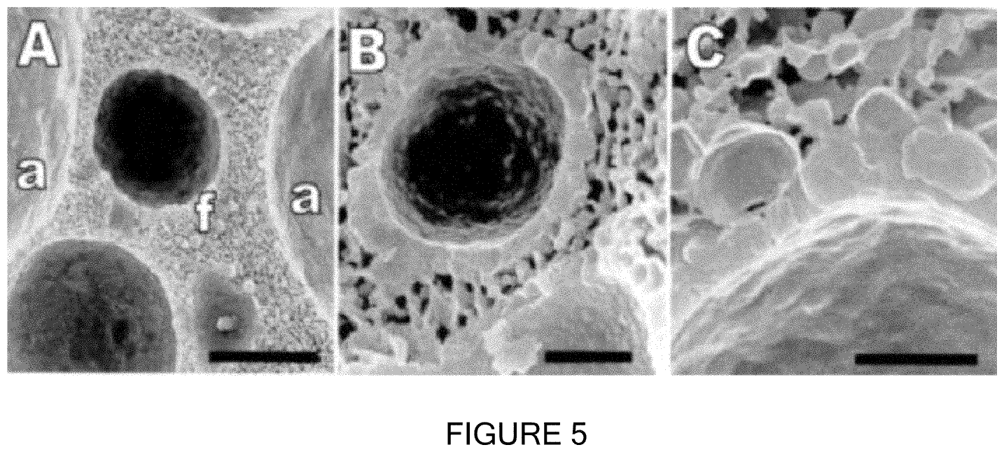

Turning now to FIG. 5, a schematic representations are shown of scanning electron micrograph (SEM) images of whipped cream, where comparison of the SEM images with the schematics shown in FIGS. 3 and 4 further provides an understanding and appreciation of the structure of whipped cream. As depicted in FIG. 5, the structure of whipped cream is shown in panels A, B, and C. Panel A provides an overview showing the relative size and prevalence of air bubbles (a) and fat globules (f); the scale bar represents 30 micrometers. Panel B shows the internal structure of an air bubble, showing a layer of partially coalesced fat that has stabilized the air bubble; where the scale bar represents 5 micrometers. Panel C provides detail of the partially coalesced fat layer, showing the interaction of the individual fat globules; where the scale bar represents 3 micrometers.

Fat partial coalescence affects things like whipped cream and ice cream structure. In canned or pressurized containers of whipping cream, a pressured gas (e.g., nitrous oxide) can be used as a propellant and whipping agent. Nitrous oxide (N₂O) is one preferred gas, as under pressure nitrous oxide dissolves in the fats in the cream, and then comes out of solution (like fizzing carbon dioxide in a soda) when the pressure is released. The bubbles of nitrous oxide whip the cream into a foam instantly as the formulation exits the pressurized container. Nitrous oxide is often used because it migrates easily into the cream and does not cause the cream to oxidize while it is in a pressurized container. Cream can have a minimum fat content of 28% to produce whipped cream with such a pressurized dispenser. When the valve of the pressurized container is opened, the cream is forced out of the nozzle by the high pressure gas. The valve of the pressurized container can include various dispensing means that varies the aperture size and cross-sectional shape of the exiting whipped cream stream to impart various profiles, textures, and character to the dispensed product.

Creamy beverages, generally, and creamy coffee beverages, in particular, can rely on finely dispersed fat (i.e., homogenized fat) to deliver mouthfeel. This emulsified fat can be delivered by various non-dairy creamers, whole or low fat milk, and whipped cream products. However, the fat found at normal levels in certain flavored coffee beverages including such products can provide insufficient mouthfeel benefits. These mouthfeel benefits can often be improved by increasing the level of fat. However, simply increasing the level of fat can create other issues, such as stability of the fat against oxidative reactions, the development of off-flavors, and the potential instability of the emulsion of the edible formulation and foaming performance thereof. Further, since certain dairy and non-dairy whipped cream products may only contain about 30% fat, delivering increased mouthfeel can require higher volumes or dosages of fats or oils. This can present issues in providing an edible formulation, that when produced as a whipped cream, can provide a stable product with a desirable mouthfeel.

Several additives can be used to enhance the stability and performance of such edible formulations. One approach to deliver mouthfeel is to use ingredients that increase the thickness or viscosity. However, increasing the viscosity does not necessarily translate into an increase in desirable mouthfeel attributes. Mouthfeel is more of a sensory perception influenced by forces distinct from those that contribute to viscosity which give the perception of thickness. Hydrocolloid gums and water-soluble starches can be used to increase beverage thickness (i.e., viscosity). However, hydrocolloid gums can only develop limited mouthfeel and can impart negative textural effects such as “sliminess” and “stringiness.” In addition, certain products that incorporate high concentrations of hydrocolloid gums can be subject to gelling upon cooling.

Water-soluble starches can also be used to increase viscosity and provide limited mouthfeel. However, the quantity of water-soluble starch needed to deliver these attributes can be to the point that more solids are added and the desired target dosage of solids in the formulation cannot be achieved. Mouthfeel, richness, creaminess, sweetness, and flavor impact can sometimes be increased by delivering a higher dosage of solids. However, such higher levels of delivered solids can require larger volumes of product to be used.

In conjunction with various thickeners, gums, and/or starches, or in place thereof, certain oils can be used with the dairy or non-dairy fats to improve emulsification and resulting whipped cream product performance. The type, blending, and amounts of such oils can improve the mouthfeel and other desired characteristics of the edible formulation. Such oils include oils and blends thereof derived from Cannabis.

The Cannabis genus of flowering plants in the family Cannabaceae produce various cannabinoids, which are one class of diverse chemical compounds that act on cannabinoid receptors in cells that can alter neurotransmitter release in the brain. Cannabinoids in the plant can be concentrated in a viscous resin produced in structures known as glandular trichomes. Over one hundred different cannabinoids can be isolated from the Cannabis plant, where such cannabinoids can be defined by the following classes of compounds: cannabigerol-type, cannabichromene-type, cannabidiol-type, tetrahydrocannabinol- and cannabinol-type, cannabielsoin-type, iso-tetrahydrocannabinol-type, cannabicyclol-type, and cannabicitran-type. All classes are derived from cannabigerol-type (CBG) compounds and differ mainly in the way this precursor is cyclized, where the cannabinoids are derived from their respective 2-carboxylic acids (2-COOH) by decarboxylation; e.g., catalyzed by heat, light, or alkaline conditions. A list of particular cannabinoids includes tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), and carmabicitran (CBT).

Cannabinoids can be separated from the plant by extraction with organic solvents. For example, hydrocarbons and/or alcohols can be used as solvents. However, certain hydrocarbons and alcohols can be flammable and/or toxic. Butane can be used for extraction, as butane evaporates quickly minimizing any residual hydrocarbon. Supercritical solvent extraction with carbon dioxide is another method. Once extracted, isolated components can be separated and characterized using wiped film vacuum distillation or other distillation methods. Cannabinoid oil extracted from cannabis can include various types of cannabinoids and fractions of cannabinoids, including various amounts of THC, THCA, CBD, CBDA, CBN, CBG, CBC, CBL, CBV, THCV , CBDV, CBCV, CBGV, CBGM, CBE, CBT, and combinations thereof. Cannabinoid oil can include other cannabis-derived compounds, including various terpenes. Such terpenes includes mixtures of monoterpenes, sesquiterpenes, and other terpenoid compounds, where examples of monoterpenes include myrcene and limonene.

Tetrahydrocannabinol (THC) can be separated from other portions or fractions of cannabinoid oil extracted from the cannabis plant. Fractions or portions of cannabinoid oil containing THC can be selected for or against to either enrich or reduce the amount of THC. In certain cases, substantially pure tetrahydrocannabinol compounds can be prepared using various methods and techniques, including those described in U.S. Pat. No. 7,923,558 to Arslantas et al. and U.S. Pat. No. 8,227,627 to Stinchcomb et al. Cannabinoid oil can be prepared from cannabis having various amounts of THC, including from about 0% THC up to about 20% THC. Alternatively, cannabinoid oil can be supplemented with additional THC fractions prepared from cannabis or from isolated THC. In certain embodiments, cannabinoid oil can be prepared having CBD as the major cannabinoid fraction and certain amounts of THC can be added thereto. It is also possible to prepare cannabinoid oil where THC is the major cannabinoid fraction. The cannabinoid oil can therefore include amounts of THC ranging from greater than 0% up to 20%, from 0.1% to 15%, from 0.3% to 12%, and from 3% to 7%. Various complementary effects can be obtained through the combination of THC with other cannabinoids, including CBD, where CBD may reduce certain undesired effects of THC including those related to sedation and intoxication.

Cannabidiol (CBD) can account for up to 40% of a cannabinoid oil extract from the cannabis plant. Cannabinoid oil containing CBD as the largest fraction of cannabinoid can be referred to as CBD oil. CBD oil can be extracted and prepared with varying amounts of other cannabinoids, including varying amounts of tetrahydrocannabinol (THC), or where there is no (THC). CBD does not have the same psychoactivity as THC. CBD can interact with different biological targets, including cannabinoid receptors and other neurotransmitter receptors. CBD can be extracted from cannabis plants, where CBD oil can be extracted from cannabis plants selectively bred to exhibit 0.3% or less THC. Alternatively, CBD oil can be processed to remove THC. For example, CBD oil can be extracted and purified to provide 0.0% THC, 70-90% CBD, in addition to other cannabinoids, terpenes and other beneficial compounds, including compounds having antioxidant activity. It is also possible to extract cannabinoid oil or CBD oil that includes various amounts of THC and/or combine or blend cannabinoid oil or CBD oil with various amounts of THC.

Ethanol and CO₂ extraction are two ways to extract cannabinoids that minimize/eliminate harmful extraction residues that may result from other processes. In CO₂ extraction, plants and/or processed plant material can be filtered through a series of chambers that control temperature and pressure. When different temperatures and units of pressure are applied to the cannabis plants and/or processed plant material, cannabinoids can be isolated at up to a 90% or more efficiency. Ethanol extraction involves introducing the solvent ethanol to the plant and/or processed plant material in order extract cannabinoids. Unlike CO₂ extraction, ethanol extraction can result in a high volume of full spectrum cannabinoid extract, including CBD. Ethanol extraction can also remove unwanted components such as chlorophyll when performed at very cold temperatures.

Once extracted, cannabinoid oil can be processed by one or more additional chromatography steps to remove unwanted plant phytochemicals from the extracted oil. Cannabinoids like CBD can have a strong interaction with certain chromatography media and can therefore proceed slower through a particular chromatographic medium than unwanted plant material like chlorophyll, for example, which can have a weak interaction with the particular chromatographic medium. Once separated, chromatographic fractions containing cannabidiol and other terpenes can be collected whereas fractions containing undesirable plant material can be disposed of.

Cannabinoid oil products can undergo what is known as decarboxylation, which involves heating the cannabinoids into a form that allows the cannabinoids to interact with the endocannabinoid system making the compound(s) more usable throughout the body. When oil extraction is decarboxylated, cannabidiolic acid (CBDA) can be converted to CBD, thereby removing the acid form so it is readily bioavailable.

Cannabinoid oil, including cannabinoid having THC and/or CBD, can be mixed with other components in the edible formulations in various ways. Various emulsions and encapsulations can be formed. In certain embodiments, the cannabinoid oil can be microencapsulated and/or nanoencapsulated using various methods. Encapsulation can improve the efficacy and bioavailability of the cannabinoid oil when consumed as part of the edible formulation. Encapsulation can also increase the half life and persistence of components of the cannabinoid oil, including THC and/or CBD. The surface of the encapsulated cannabinoid oil can also be modified with various hydrophilic polymers or ligands to increase absorption and uptake. Surface modification, including the coupling of small molecules on the surface of such encapsulations, can also increase cellular uptake and interaction. Examples of encapsulation include those described in Kumari, A., Singla, R., Guliani, A., & Yadav, S. K. (2014). Nanoencapsulation for drug delivery. EXCLI journal, 13, 265-286 and the examples described in Patra, J. K. et al. (2018). Nano based drug delivery systems: recent developments and future prospects. J. of Nanobiotechnology, 16:71.

Dispensing the edible formulations provided by the present technology from pressurized containers is one means to generate a whipped cream product on-demand that has suitable foam characteristics and that is shelf-stable. Various gaseous components can be used to pressurize the containers described herein. Suitable gases include nitrogen, N₂O (nitrous oxide), hydrogen, carbon dioxide, argon, and combinations thereof. Use of nitrous oxide and propellants including nitrous oxide can help the formulation become more emulsified as the nitrous oxide dissolves in the composition and foams out of the composition upon dispensing of product. Use of nitrous oxide can further work in conjunction with the fat and oil content of the edible formulation to develop and maintain its creaminess/mouthfeel.

Various amounts of oils and blends thereof can be made for the edible formulation including the cannabinoid oil (e.g., CBD oil) to provide an improved whipped cream product. Various percentages of oil derived from cannabis can be included, for example, where certain embodiments include from 0.01% to 10% CBD oil. Other embodiments include from 0.05% to 5% CBD oil, 0.1% to 2.5% CBD oil, and 0.5%-1% CBD oil.

The present technology can therefore provide edible formulations that include an aqueous component, a lipid component including a triglyceride, and a cannabis-derived oil. Certain embodiment include where the edible formulation is comprised by from about 55 wt. % to about 65 wt. % of the aqueous component, from about 25% to about 40% of the lipid component, and from about 0.1 wt. % to about 10 wt. % of the cannabis-derived oil. The edible formulation can be in the form of an emulsion having a continuous phase including the aqueous component and a dispersed phase including the lipid component and the cannabis-derived oil.

The lipid component can be surrounded by at least one layer including a phospholipid. For example, where at least a portion of the lipid component is dairy-based, the lipid component can include a milk fat globule membrane composed of lipids and proteins that surrounds a milk fat globule. In particular embodiments, the milk fat globule is surrounded by a phospholipid trilayer containing associated proteins, carbohydrates, and lipids. The milk fat globule membrane can make up about 2% to 6% of the total milk fat globule and can provide a source of phospholipids, accounting for a majority of total milk phospholipids. In contrast, the inner core of the milk fat globule can be composed predominantly of one or more various triglycerides.

Various lipid components can include one or more triglycerides derived from various sources, including triglycerides derived from animals and/or plants. The lipid component including the triglyceride can be animal-derived. Examples of animal-derived triglycerides include where the triglyceride includes glycerol esterified with fatty acids such as myristic acid, palmitic acid, stearic acid, palmitoleic acid, oleic acid, linoleic acid, alpha-linolenic acid, vaccenic acid, and combinations thereof. The lipid component including the triglyceride can be a plant-derived. Examples of plant-derived triglycerides include where the triglyceride includes glycerol esterified with fatty acids such as caprylic acid, decanoic acid, lauric acid, myristic acid, palmitic acid, oleic acid, stearic acid, linoleic acid, arachidic acid, behenic acid, lignoceric acid, alpha-linolenic acid, and combinations thereof.

Various oils and combinations of oils derived from cannabis can be used in the edible formulation. Embodiments include where the cannabis-derived oil includes one or more of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran. Particular embodiments include where the cannabis-derived oil includes cannabidiol and can include where the cannabis-derived oil consists essentially of cannabidiol and where the cannabis-derived oil consists of cannabidiol. Other embodiments include where the cannabis-derived oil includes cannabidiol as the largest fraction thereof. Certain embodiments include where cannabis-derived oil has from about 70 wt. % to about 90 wt. % cannabidiol. Embodiments further include where the cannabis-derived oil includes less than about 0.3% THC and some embodiments include where the cannabis-derived oil includes about 0.0% THC.

Edible formulations can be in the form of a colloid having a continuous phase including the edible formulation and a dispersed phase including a gas. The gas can comprise, consist essentially of, or consist of nitrous oxide, where as described herein, the nitrous oxide can be soluble in the lipid component including the triglyceride. In this way, the nitrous oxide comes out of solution (like fizzing carbon dioxide in a soda) when pressure is released and bubbles of nitrous oxide can whip the edible formulation into a foam instantly as the edible formulation exits a pressurized container, for example. Nitrous oxide can migrates easily into the lipid component and does not cause the lipid component to oxidize while it is in a pressurized container.

Edible formulations provided herein can further include one or more food additives to provide or enhance certain characteristics. Examples of additives include the addition of one or more sweeteners, non-nutritive sweeteners, flavoring agents, colorants, emulsifiers, thickeners, and/or preservatives. Sweeteners include simple sugars, also called monosaccharides, including glucose, fructose, and galactose, compound sugars, also called disaccharides or double sugars, which include two monosaccharides joined by a glycosidic bond, such as table sugar (glucose+fructose), lactose (glucose+galactose), and maltose (two molecules of glucose). Non-nutritive sweeteners can include acesulfame potassium, aspartame, cyclamate, mogrosides, saccharin, stevia, sucralose, and/or sugar alcohols. Natural flavoring substances can be added, including flavoring substances obtained from plant or animal raw materials, by physical, microbiological, or enzymatic processes, and can be either used in their natural state or processed. Nature-identical flavoring substances can be included, including those obtained by synthesis or isolated through chemical processes, which are chemically and organoleptically identical to flavoring substances naturally present in products intended for human consumption. Artificial flavoring substances can be included in the formulation. The various flavoring substances can include flavor profiles derived from or intended to replicate apple, butter, banana, almond, cherry, cinnamon, grape, orange, pear, pineapple, vanilla, mint, wintergreen, among other flavors. Natural and artificial coloring agents can also be included. Examples of emulsifiers include egg yolk (in which the main emulsifying agent is lecithin), soy lecithin, phosphates, mono- and diglycerides, cellulose, etc. Thickeners include those based on polysaccharides (starches, vegetable gums, and pectin) and proteins. Preservatives include sorbic acid and sorbates, benzoic acid and benzoates, sulfur dioxide and sulfites, nitrates and nitrites, lactic acid, propionic acid and propionates, antioxidants including ascorbic acid and ascorbates, tocopherols, ethanol, etc.

Certain manufacture of the edible formulations provided herein include where the edible formulation is packaged in a pressurized container. The pressurized container can be pressurized with a gas including nitrous oxide. For example, the pressurized container can be formed of metal and have a dispensing nozzle can be configured with various aperture sizes and cross-sectional shapes so that various profiles, shapes, textures, and character is imparted to the dispensed edible formulation.

Methods of making the various edible formulations are also provided by the present technology. In certain embodiments, methods of making an edible formulation include providing an edible formulation as described herein and generating a colloid having a continuous phase including the edible formulation and a dispersed phase including a gas. This allows the edible formulation to be provided as a whipped dairy or non-dairy topping.

Example edible formulations constructed in accordance with the present technology include the following embodiments.

### First Embodiment

1 pint heavy cream (boxed whipping cream is all right but often includes additives)
1 ounce cannabinoid oil (e.g., CBD oil, including primarily CBD as well as other cannabinoids)
1 tablespoon maple syrup (optional)
¼ teaspoon vanilla extract
CO₂ canister and cartridge or hand mixer
   Instructions for the canister: Measure ingredients based on the capacity of your canister into a measuring cup. Stir until combined. Pour into device, charge with two CO2 chargers. Dispense cream at will.
   Instructions for a hand mixer: In a large bowl (you want room to work) stir ingredients until mixed. Whip on medium with hand mixer until triple in size. Stop when it's airy and silky and still has movement; any further and it will begin to harden and make butter. If that's what you want, roll on.

### Second embodiment

1 pint heavy cream (boxed whipping cream is all right but often includes additives)
1 ounce cannabis oil (e.g., cannabinoid oil, including primarily CBD as well as other cannabinoids)
1 tablespoon maple syrup (optional)
¼ teaspoon vanilla extract
CO₂ canister and cartridge or hand mixer
   Instructions for the canister: Measure ingredients based on the capacity of your canister into a measuring cup. Stir until combined. Pour into device, charge with two CO2 chargers. Dispense cream at will.
   Instructions for a hand mixer: In a large bowl (you want room to work) stir ingredients until mixed. Whip on medium with hand mixer until triple in size. Stop when it's airy and silky and still has movement; any further and it will begin to harden and make butter. If that's what you want, roll on.

### Third Embodiment

4-8 grams of cannabinoid oil (e.g., CBD oil, including primarily CBD as well as other cannabinoids)
1 cup heavy cream
1 teaspoon vanilla extract (optional)
1 tablespoon confectioners' sugar
   Instructions: 1. Add the cream and cannabis to a pan over medium heat in a double boiler. 2. Allow the cannabis cream to simmer for about an hour. 3. Allow to cool and then pour into an airtight container in the refrigerator until cold. 4. Using a large bowl, whip cream until peaks hold there form or desired consistency. Beat in vanilla and sugar. Do not over-beat as it will become lumpy.

### Fourth Embodiment

⅛ oz of cannabinoid oil (e.g., CBD oil, including primarily CBD as well as other cannabinoids)
1 cup of half and half
½ teaspoon vanilla extract
1 tablespoon sugar
¼ cup of espresso
1 tablespoon pumpkin puree
1 teaspoon pumpkin spice

Fifth embodiment: Coconut Whipped Cream

Vegan, gluten-free, grain-free, no bake/raw, nut-free, oil-free, refined sugar-free, soy-free. A fluffy whipped cream can be created by using a can of full-fat coconut milk. The can of coconut milk can be chilled for at least 24 hours before to ensure the white coconut cream solidifies. Yield 1 cup (250 mL), Prep time 10 Minutes, and Cook time 0 Minutes.

### Ingredients:

1 (14-ounce/400 mL) can full-fat coconut milk, chilled for 24 hours
1 to 2 tablespoons sweetener (maple syrup, powdered sugar, cane sugar, etc), to taste
1 vanilla bean, scraped or ½ teaspoon pure vanilla extract (optional)
½ oz cannabinoid oil, including primarily CBD as well as other cannabinoids

### Directions:

Chill the can of coconut milk in the fridge for at least 24 hours. About 1 hour before making the coconut whip, chill a mixing bowl in the freezer. After chilling the can, open the can and scoop the solid white coconut cream into the bowl. Discard the coconut water or save it for another use (such as coconut water ice cubes). Add the CBD oil. Using an electric hand mixer or a stand mixer with the whisk attachment, beat the cream until fluffy and smooth. Add in sweetener to taste and vanilla. Return whipped cream to fridge until ready to use. It will firm when chilled and soften at room temperature. This will keep in the fridge in a sealed container for up to 1 week or you can freeze it in an airtight freezer- safe bag for up to 1 month. After chilling in the fridge, allow it to sit at room temperature until it softens slightly and then you can re-whip it as needed.

### Sixth Embodiment

Ingredients
Coconut cream, cannabinoid oil, water, sugar/glucose-fructose, mono- and di-glycerides, carrageenan, xanthan gum, nitrous oxide (pressure dispensing agent).
Lactose Free, Dairy Free, Gluten Free, Cholesterol Free, Certified Vegan, Certified Non GMO Ingredients

### Seventh Embodiment

Cream, CBD oil, water, sugar/glucose-fructose, buttermilk powder, mono and diglycerides, carrageenan, natural flavor, nitrous oxide (pressure dispensing agent). Contains: milk.

### Eight Embodiment

Edible formulation for non-dairy whipped cream: water, hydrogenated vegetable oil (including coconut and palm kernel oils), high fructose corn syrup, corn syrup, skim milk, light cream (less than 2%), cannabinoid oil, sodium caseinate, natural and artificial flavor, xanthan and guar gums, polysorbate 60, sorbitan monostearate, sodium polyphosphate, and beta carotene (as a coloring), with Nitrous Oxide as propellant.

### Ninth Embodiment

Edible formulation for dairy whipped cream: Nonfat Milk, Cream, Cannabinoid Oil, Sugar, Corn Syrup, Maltodextrin, Inulin (Chicory Extract), Cellulose, Mono- and Diglycerides Polysorbate 80, Artificial Flavors, Carrageenan, with Nitrous Oxide as propellant.

The various edible formulations described herein can be packaged into pressurized containers for dispensing a foamed product therefrom having improved mouthfeel and calming properties.

### Tenth Embodiment

An edible formulation includes a cannabis-derived oil within a pressurized container. The container is pressurized with nitrous oxide. The cannabis-derived oil includes one or more of the cannabis-derived oils described herein. The pressurized container including the edible formulation of cannabis-derived oil can include various additional components, such as one or more sweeteners, non-nutritive sweeteners, flavoring agents, colorants, emulsifiers, thickeners, preservatives, and combinations of such components. It is also possible to include a lipid component having one or more triglycerides within the edible formulation packaged within the pressurized container. Embodiments include a pressurized container comprising an edible formulation including a cannabis-derived oil, wherein the container is pressurized with nitrous oxide.

Benefits and advantages of the present technology include combined, broadened, and complementary relaxing and calming effects of cannabinoids and antioxidant activities of CBD oil that can contribute to creamy mouthfeel, richness, and flavor impact of edible formulations for whipped cream. The formulations can alleviate stress and pain and the mixture of dairy-based and/or non-dairy based fats and oils can improve the uptake and absorption of CBD in the CBD oil, as well as other cannabinoids present in the CBD oil. CBD oil can bring the effects of CBD itself as well as additional cannabinoids that can modulate anxiety, cognition, movement disorders, and pain in beneficial ways.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An edible formulation comprising:
an aqueous component;
a lipid component including a triglyceride; and
a cannabis-derived oil.

2. The edible formulation of claim 1, comprised by from about 55 wt. % to about 65 wt. % of the aqueous component, from about 25% to about 40% of the lipid component, and from about 0.1 wt. % to about 10 wt. % of the cannabis-derived oil.

3. The edible formulation of claim 1, wherein the edible formulation is in the form of an emulsion having a continuous phase including the aqueous component and a dispersed phase including the lipid component and the cannabis-derived oil.

4. The edible formulation of claim 1, wherein the lipid component is surrounded by at least one layer including a phospholipid.

5. The edible formulation of claim 1, wherein the lipid component is animal-derived.

6. The edible formulation of claim 1, wherein the triglyceride includes glycerol esterified with a fatty acid selected from a group consisting of: myristic acid, palmitic acid, stearic acid, palmitoleic acid, oleic acid, linoleic acid, alpha-linolenic acid, vaccenic acid, and combinations thereof.

7. The edible formulation of claim 1, wherein the lipid component is a plant-derived.

8. The edible formulation of claim 1, wherein the triglyceride includes glycerol esterified with a fatty acid selected from a group consisting of: caprylic acid, decanoic acid, lauric acid, myristic acid, palmitic acid, oleic acid, stearic acid, linoleic acid, arachidic acid, behenic acid, lignoceric acid, alpha-linolenic acid, and combinations thereof.

9. The edible formulation of claim 1, wherein the cannabis-derived oil includes a member selected from the group consisting of: tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, and combinations thereof.

10. The edible formulation of claim 1, wherein the cannabis-derived oil includes cannabidiol.

11. The edible formulation of claim 1, wherein the cannabis-derived oil includes cannabidiol as the largest fraction thereof.

12. The composition of claim 1, wherein cannabis-derived oil includes from about 70 wt. % to about 90 wt. % cannabidiol.

13. The composition of claim 1, wherein the cannabis-derived oil includes less than about 0.3% THC.

14. The composition of claim 1, wherein the cannabis-derived oil includes about 0.0% THC.

15. The edible formulation of claim 1, wherein the edible formulation is in the form of a colloid having a continuous phase including the edible formulation and a dispersed phase including a gas.

16. The edible formulation of claim 15, wherein the gas includes nitrous oxide.

17. The edible formulation of claim 1, further comprising a member selected from a group consisting of: a sweetener, a non-nutritive sweetener, a flavoring agent, a colorant, an emulsifier, a thickener, a preservative, and combinations thereof.

18. The edible formulation of claim 1, wherein the edible formulation is packaged in a pressurized container.

19. The edible formulation of claim 18, wherein the pressurized container is pressurized with a gas including nitrous oxide.

20. A method of making an edible formulation, the method comprising:
providing an edible formulation including an aqueous component, a lipid component including a triglyceride, and a cannabis-derived oil; and
generating a colloid having a continuous phase including the edible formulation and a dispersed phase including a gas.

21. A pressurized container comprising an edible formulation including a cannabis-derived oil, wherein the container is pressurized with nitrous oxide.
